(19) United States
(12) Patent Application Publication
(10) Pub. No.: US 2007/0032840 A1
(43) Pub. Date: Feb. 8, 2007

(54) Portable electric stimulator for wrinkles with interchangeable microelectrodes applicator

(76) Inventor: Francesco Peluso, Salerno (IT)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(21) Appl. No.: 11/581,119

(22) Filed: Oct. 13, 2006

Related U.S. Application Data
(63) Continuation of application No. PCT/IT05/00213, filed on Apr. 14, 2005.

(51) Int. Cl. A61N 1/00

(52) U.S. Cl. 607/66

(57) ABSTRACT

Portable electric stimulator for wrinkles with interchangeable microelectrodes applicator Apparatus that generates electrical micro-currents realized through a wave of 6000 Hz modulated by a emi-sinusoidal and uni-directional 100 Hz wave applied to the skin by a set removable heads with electrodes needles shaped, from the lodging point or easy to insert by a simple lateral push. The apparatus aims to be of small dimensions, portable, cheap and above all of simple and safe operation so that whoever is able to use it for the treatment of wrinkles due to both to cutaneous aging and expression or compression wrinkles, at home or wherever a plug is available.

### CROSS-REFERENCE TO RELATED APPLICATIONS

This patent application is a continuation of copending International Patent Application No. PCT/IT2005/000213, filed Apr. 14, 2005, which designates the U.S.

### TECHNICAL FIELD

The apparatus object of the above mentioned invention falls within the field of portable devices for aesthetic treatments and cure of the body in particular wrinkles, stretch marks by using modulated micro-currents of vlf (low frequency).

### TECHNICAL STATUS

It is well-known that as the age increases, the dermal layer grows thin and the degree of generation of the collagen diminishes, the reticular and fibrous structure grows determining a wrinkle crumple of the skin and a loss of elasticity.

Different kinds of realizations in treating wrinkles are known, for example in WO 98/33558, titled “Method and apparatus for treating wrinkles in skin using radiation,” that involves the use of beam of pulsed continuous wave laser or incoherent radiation of between 1.3 and 1.8 microns to a targeted dermal region between 100 microns and 1.2 millimetres below a wrinkle in the skin. In EP 1457234(A2), titled “Therapeutic light source and method,” comprises a rigid arrays of Led (LF, LL, LR) movably connected together and with emission of spectra substantially with an output intensity of at least 10 Mw/cm². In EP 0387176(A1), titled “Apparatus for the treatment of wrinkles with one-way, electrical, few micro-amperes, low frequency waves and ionisable cosmetic products,” comprises a rotating movable head, which is interchangeable or fixed, with an edge portion is conducting and thin enough to penetrate in the flanks between the two furrows of the wrinkle. The low-frequency waves are between 0-400 Hz.

In EP 091853(A1), titled “Method for skin treatment and apparatus applying this method”. This method using a pulse generator with a needle which is introduced immediately under the skin and in substantially parallel relationship to the wrinkle, and in making pass through the said needle a weak current of low frequency comprised between about 100 and 500 cps.

In WO 99/17690, titled “Systems and method for electrosurgical tissue contraction.” In WO 99/26546, titled “Systems and method for electrosurgical treatment of the skin,” applies high frequency (RF) electrical energy to one or more electrode terminals sufficient to heated collagen fibres in the range of about 45 to 90° C.

In the current application one takes advantage of the principle of micro-currents in order to generate the perspiration stage to the dermal level. This phase is exclusively stimulated inside the wrinkle you wish to treat thanks to the specific applicator provided with microelectrodes which only supports the contact with the inside of the wrinkle. The perspiration stage is provoked by the heat generated by micro-currents produced, heat that microscopically generates a proliferation of fibroblasts cells which allows a localized new making of connecting tissue localized under the wrinkle, creating therefore a pushing effect from the bottom. The treated cells in fact use the thermic energy in excess immediately available in order to promote the essential and fundamental metabolic dermal processes, such as the proteins synthesis, or the essential new-synthesis both of collagen and elastin essential to the process of rebuilding of the collapsed tissues under the wrinkle, so soon after the first application you get the effect of levelling of the wrinkle due to the wrinkle rebuilding just under it.

The use of micro-currents widely supported by documents within the medical field (Wing, 1973) and Ghersetich et al., “Ultrastructural study of hyaluronic acid before and after the use of a pulsed electromagnetic field, electrorydesis, in the treatment of wrinkles,” Int. J. Dermatol., 33 (9): 661-663 (1994). It allows therefore to activate the local metabolism for the rebuilding of youth molecules which are the collagen that gives compactness to the skin, and elastin which give elasticity to tissues.

It is well-known an apparatus used in centres for aesthetic and cure of the body made up of a container, to whose inside you can find the operative electronics, put on a plan and connected to the container through a cable, a handle (an handling instrument) bringing needles electrodes of changeable number, according to models which properly apply waves modulated of vlf (Low frequency), also combined with sources of light sent out from a laser for the treatment of wrinkles due to cutaneous aging.

It is known that electrical stimuli provoke a more or less intense painful feeling in function of the kind of waves and frequencies used. The use of bi-directional currents with frequencies comprised between 2500 and 8000 Hz (MF) assures the best tolerance of the human being. The above mentioned apparatus works on a frequency of 6000 Hz modulated on low frequency with a modulating sinusoidal wave. From 50-200 Hz. The apparatus proves to be of not small dimensions, expensive, and above all it is exclusively used in beauty centres which have a specialized staff able 5 to best use such apparatus.

### AIMS AND ADVANTAGES OF THE INVENTION

The main aim of the current invention is to propose an apparatus that generates electrical micro-currents realized through a wave of 6000 Hz modulated by a emi-sinusoidal and uni-directional 100 Hz wave applied to the skin by electrodes. The apparatus aims to be of small dimensions, portable, cheap and above all of simple and safe operation so that whoever is able to use it for the treatment of wrinkles due to both to cutaneous aging and expression or compression wrinkles, at home or wherever a plug is available.

Together with the previous one another aim of such invention is the realization of a set removable heads from the lodging point or easy to insert by a simple lateral push. Another aim of the current invention together with the previous ones is to 20 carry out three kinds of heads respectively to five electrodes needles shaped, to seven electrodes and eleven electrodes needles shaped for a selective use on more or less expanded wrinkles.

A further aim of the current invention together with the previous ones is to make on each single head a movable protection pointed hood.

Another aim of the invention together with the previous ones is to make the apparatus provided with an independent electrical feeding by means of reloadable or not reloadable batteries.

A clinical study was conducted at the University of Naples which tested the efficacy and safety of the apparatus in accordance to the current invention.

### DRAWINGS DESCRIPTION AND WAY OF USE OF THE INVENTION

Further characteristics of the found invention will result mainly from the description of favourite forms of execution here illustrated for a recommending but not limitative purpose from drawings here enclosed:

FIG. 1 shows the apparatus in its complex

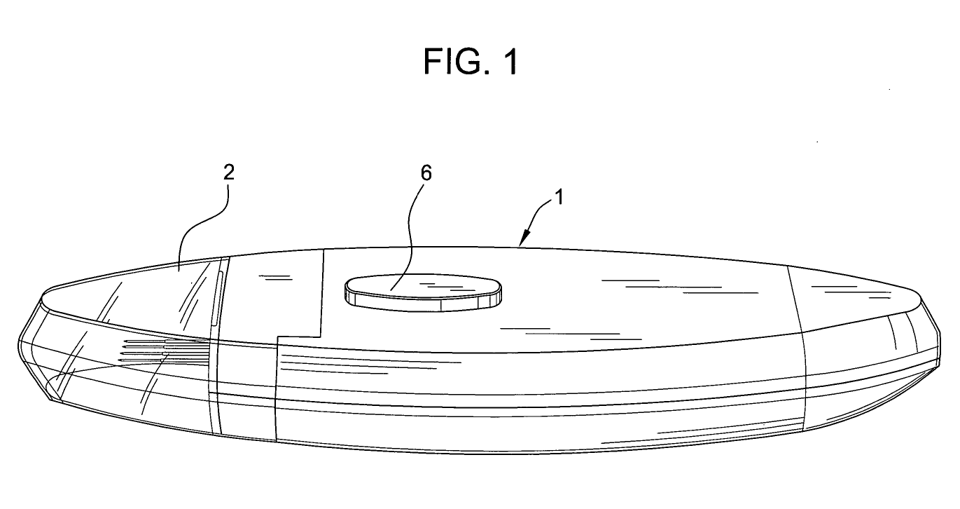

FIG. 2 shows the total part of the apparatus showing the electrodes needles shaped, without the protection cap.

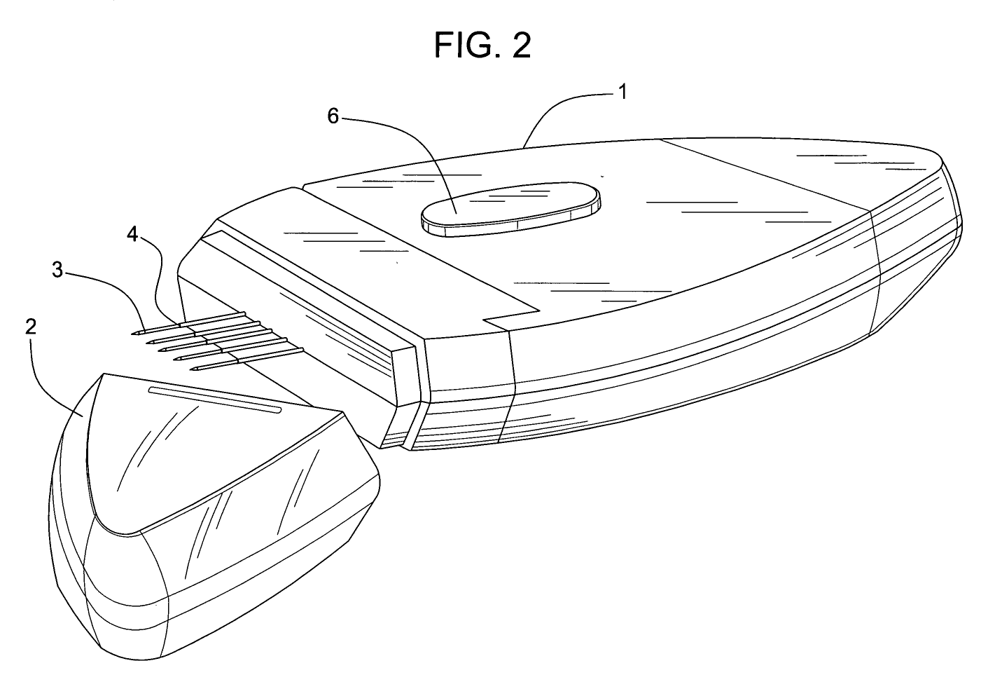

FIG. 3 shows a blocks operating outline of the electronic chip. In compliance to drawings the stimulating apparatus, object of the current invention, for a first step of realization is made up as follows: a plastic container elliptic shaped 1 which comprises on the front, a transparent plastic cap 2 triangular shaped with round edges to protect the electrodes 3, equipped with shock-absorbers 4 adaptable to the wrinkle configuration which come out from a removable front portion stick connected through an sliding guide to the remaining body of the apparatus with no locked device; the applicator can be removed, (preferably always with the protecting cap 2 in order to avoid accidental damages to electrodes) acting on its lateral side through a hand light push, making a lateral sliding movement, until this comes out from its centre; to reassemble or replace the applicator with a different one place it with the cover inserted in sliding guide and let it slide with a light push in the opposite direction to replace one until it locks; on the back you can find the gudgeon spin (not shown) to connect the transformer 12v continuous current or other kind of tensions, to the plug, usually 220v or 110v; on the top of the stimulating apparatus you can find a conformed element, coming out from the plastic container which allows an elliptic shaped metallic contact 6; inside the plastic container you can find an electronic chip.

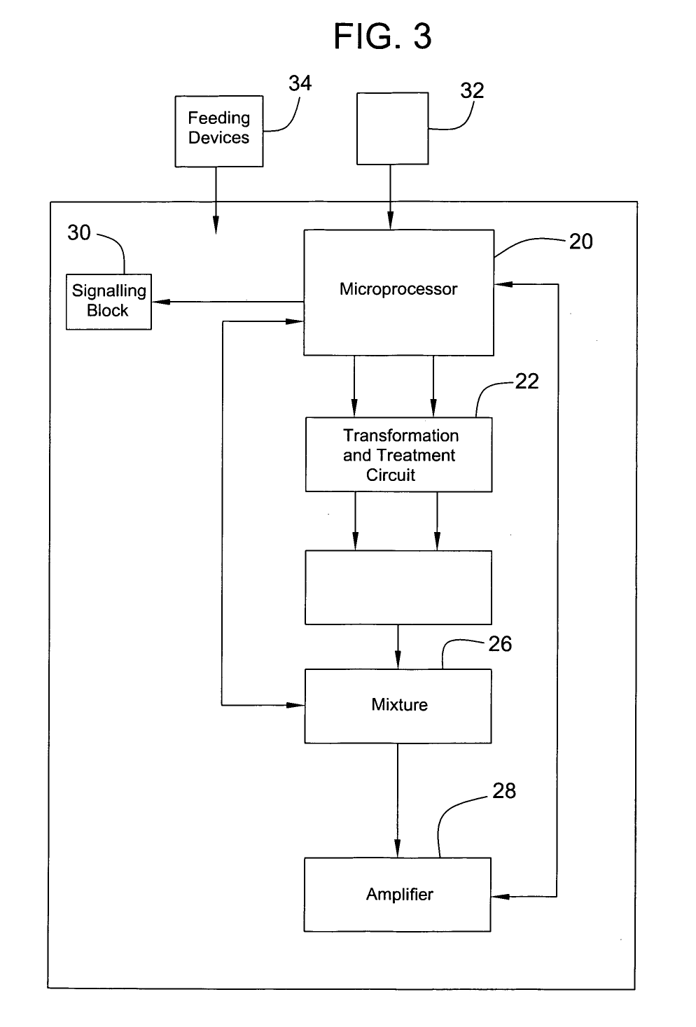

Referring to the blocks operating outline of FIG. 3, on the chip there is the microprocessor represented by block 20 that generates two different signals by a square wave, the first one of 6000 Hz and the second one of 50 Hz; these two signals deal with the block of transformation and treatment circuits 22 where they are transformed in a sinusoidal wave and the signal to 50 Hz is brought to 100 Hz. The signals deals with the mixer block 26, where they are mixed in order to get a single wave to low amplitude that catches up the amplifier block 28 which supplies to increase the amplitude according the ordering signal that directly comes from the microprocessor 20 and concerns the time the patient physically holds on his skin the block made up of microelectrodes applicator. The signalling block 30 is ordered by the signal directly coming from the microprocessor 20, and produces a sound signal that increases of intensity as the time of contact of the block 28 to the skin and of the contact oval 6 increases. This is showed in picture 3 through the block 32. Block 34 which is represented in picture 3 represents feeding devices.

Obviously all the functions of the blocks diagram of FIG. 3 could be contained also within a single electronic component purposely realized or anyway available on trade.

The maximum output current varies between 4 and 8 Ma (0.1-10 k) Ohm.

The maximum output tension is 4-6v (1000 Ohm). The shape of the output wave is sinusoidal and is modulated in its amplitude by a emi-sinusoidal and a one-directional 100 Hz current.

Another favourite shape of realization of the current invention is made up of seven electrodes to be employed on longer wrinkles in order to spend less time to get the result.

Another variant of the current invention is provided with the removable applicator to be used on particularly long wrinkles and eventually also on the skin stretch marks. Another favourite kind of realization of such invention is provided with the apparatus which has an independent feeding by rechargeable batteries and in the back the connection to the battery charger can be found. Another kind of realization of such apparatus is made up of a memory component that allows a completely automatic operation once you set up the time according to the kind of face-skin or imperfection to treat. This allows an easier, more comfortable and safer use.

Another kind of realization the apparatus can have a geometric shape of the container and of the protection cover, rather different, in fact they can be ergonomic, rectangular or trapezium shaped, cylindrical or even semicircular.

The use of the stimulating apparatus is very easy:

After having connected the apparatus throughout the transformer in the plug, remove the microelectrodes protection cover being careful not to break off or fold them, since the apparatus is not provided of a push-5 button, as this can be made laying the microelectrodes on the wrinkle and holding a finger on the specific contact oval 6; while it can be automatically turned off by taking away the microelectrodes from the wrinkle or keeping away the finger from the oval contact 6. After having slightly laid the microelectrodes on the wrinkle furrow on the parallel way within a few seconds the apparatus starts with the distribution of the current and begins to send out a sound intermittent signal that becomes more and more frequent as the time of rest and the intensity of the sent out current increase. As the current intensity is not longer pleasant (usually within a time that varies between 3 and 10 seconds according to the kind of skin) the microelectrodes are raised from the contact with the wrinkle and after a few seconds you act again on the same wrinkle in order to carry on the treatment. The operation is repeated more and more times on the same wrinkle until a total time of 20-30 seconds for each single wrinkle or part of the same is reached.

In case of long or particularly long wrinkle and skin stretch marks an apparatus respectively made up of seven or eleven electrodes is used and the previous operation is repeated more and more times until the whole length of the wrinkle or stretch mark is covered.

The above mentioned apparatus proves to be able to attenuate face wrinkles in a totally natural and not invasive way. In fact it stimulates the natural production of collagen and elastin taking advantage of the principle of the perspiration inflammatory reaction. The results produced are not permanent, but together with a proper maintenance programme anyway they can last for months. This proves to be comfortable, and little expensive as for the first time whoever suffers from these aesthetic bodily defects is given the opportunity to easily and safely use such apparatus at home.

As described in the lines above the apparatus reaches the expected purposes.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A stimulator apparatus for skin wrinkles comprising:
an electrode;
a first generator generating a first signal having a first frequency;
a second generator generating a second signal having a second frequency lower than said first frequency;
a mixer mixing said first signal and said second signal;
a microprocessor sending an ordering signal to increase the amplitude of said mixed first and second signal as the time the stimulator apparatus contacts the skin increases.

2. The stimulator apparatus as claimed in claim 1 further comprising a transformation circuit transforming said first signal and said second signal from a square wave to a sinusoidal wave.

3. The stimulator apparatus as claimed in claim 1 wherein said first frequency is between 2500 and 8000 Hz.

4. The stimulator apparatus as claimed in claim 1 wherein said first frequency is approximately 6000 Hz.

5. The stimulator apparatus as claimed in claim 1 wherein said second frequency is between 0 and 400 Hz.

6. The stimulator apparatus as claimed in claim 1 wherein said second frequency is between 50 and 200 Hz.

7. The stimulator apparatus as claimed in claim 1 wherein said second frequency is approximately 50 Hz.

8. The stimulator apparatus as claimed in claim 1 further comprising a treatment circuit increasing frequency of said second signal from said second frequency to a third frequency.

9. The stimulator apparatus as claimed in claim 8 wherein said third frequency is approximately 100 Hz.

10. The stimulator apparatus as claimed in claim 1 further comprising a container having a cap.

11. The stimulator apparatus as claimed in claim 1 further comprising shock-absorbers adaptable to configuration of the wrinkles.

12. The stimulator apparatus as claimed in claim 1 wherein said electrode is mounted on a removable head.

13. The stimulator apparatus as claimed in claim 12 further comprising a sliding guide connecting said removable head to remainder of the apparatus.

14. The stimulator apparatus as claimed in claim 1 further comprising a gudgeon spin.

15. A method for treatment of skin wrinkles of a patient comprising the steps of:
a) providing a stimulator apparatus having an electrode;
b) contacting said stimulator apparatus to the skin of the patient;
c) generating a first signal by a square wave having a first frequency;
d) generating a second signal by a square wave having a second frequency lower than said first frequency;
e) mixing said first signal and said second signal;
f) providing a microprocessor; and
f) increasing the amplitude of said mixed first and second signals according to an ordering signal from said microprocessor.

16. The method as claimed in claim 15 wherein said step of increasing the amplitude of said mixed first and second signals increases the amplitude as the time said stimulator apparatus contacts the skin increases.

17. The method as claimed in claim 15 further comprising the step of transforming said first signal and said second signal from a square wave to a sinusoidal wave.

18. The method for treatment of skin wrinkles as claimed in claim 15 wherein said first frequency is between 2500 and 8000 Hz.

19. The method for treatment of skin wrinkles as claimed in claim 15 wherein said first frequency is approximately 6000 Hz.

20. The method for treatment of skin wrinkles as claimed in claim 15 wherein said second frequency is between 0 and 400 Hz.

21. The method for treatment of skin wrinkles as claimed in claim 15 wherein said second frequency is between 50 and 200 Hz.

22. The method for treatment of skin wrinkles as claimed in claim 15 wherein said second frequency is approximately 50 Hz.

23. The method for treatment of skin wrinkles as claimed in claim 15 further comprising the step of increasing the frequency of said second signal from said second frequency to a third frequency prior to said step of mixing said first signal and said second signal.

24. The method for treatment of skin wrinkles as claimed in claim 23 wherein said third frequency is approximately 100 Hz.

25. The method for treatment of skin wrinkles as claimed in claim 15 further comprising the step of producing a sound signal that increases in intensity as the time of said stimulator apparatus contacts the skin increases.

26. A stimulator apparatus for skin wrinkles comprising:
an electrode;
a generator generating a first signal having a first frequency and a second signal having a second frequency lower than said first frequency;
a mixer mixing said first signal and said second signal;
a microprocessor sending an ordering signal to increase the amplitude of said mixed first and second signal as the time the stimulator apparatus contacts the skin increases.
